(19) United States
(12) Patent Application Publication
(10) Pub. No.: US 2005/0163874 A1
(43) Pub. Date: Jul. 28, 2005

(54) Data analysis method

(76) Inventor: Blagoy Petrov Alexiev, Sofia (BG)

Correspondence Address: AKERMAN SENTERFITT, P.O. BOX 3188, WEST PALM BEACH, FL, 33402-3188, US

(21) Appl. No.: 10/505,743

(22) PCT Filed: Sep. 19, 2002
(86) PCT No.: PCT/GB02/00023

(51) Int. Cl. A61K 35/78; A61K 31/704; A61K 31/56

(52) U.S. Cl. 424/757; 514/170; 514/26

(57) ABSTRACT

The invention relates to a pharmacological substance based on biologically active substances obtained from the plant Tribulus terrestris L. to be used as an agent reducing blood sugar, improving blood circulation and especially blood circulation in veins and capillaries of the limbs of diabetic patients, reducing bad cholesterol level, increasing good cholesterol concentration, maintaining cardiovascular and liver functioning with an additional prophylactic or healing effect on the immune system and the immune resistance. The pharmacological substance represents a combination of bio-active trivalent chromium and a complex of steroid saponins obtained from the plant Tribulus Terrestris l. and consisting of furostanols, spirostanols, sapogenins, sterols, flavonoids and other biologically active substances typical of this plant.

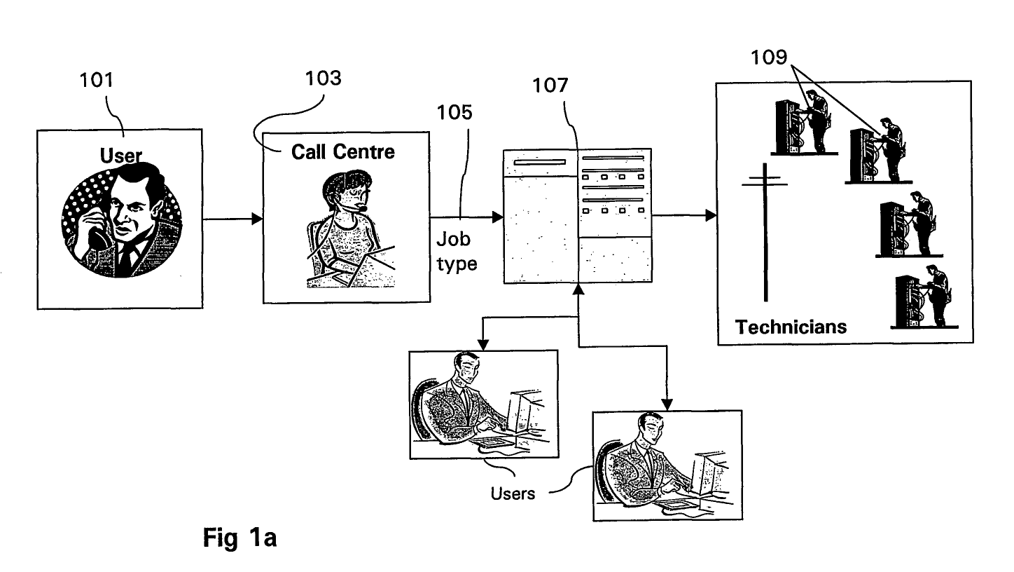

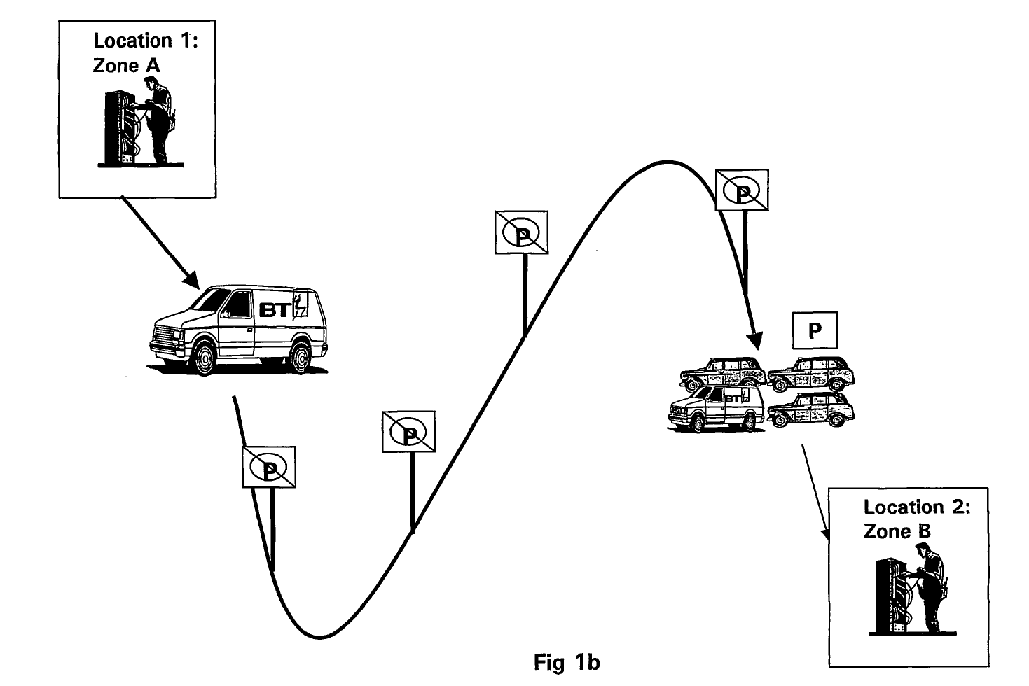

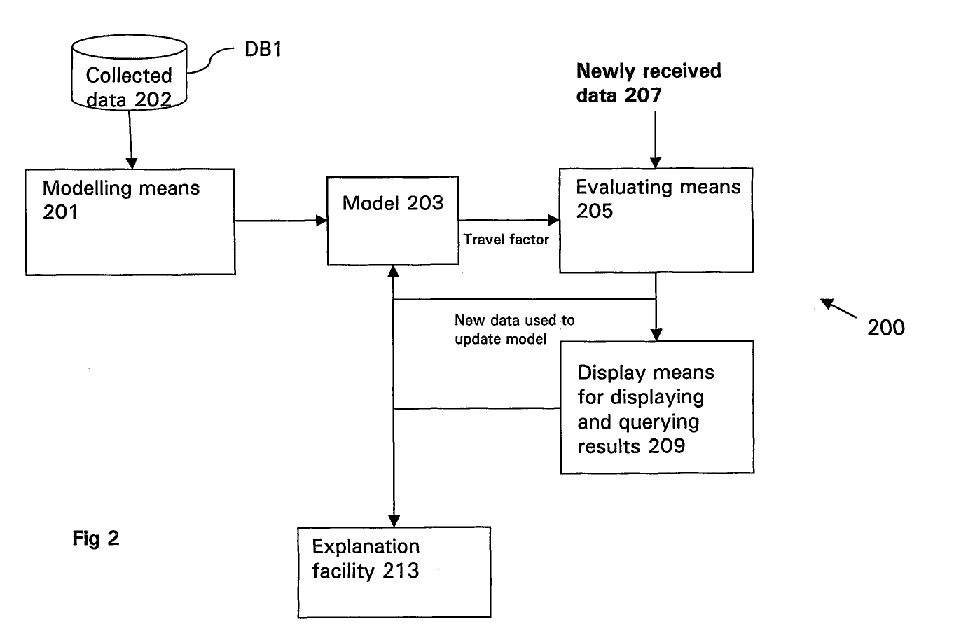

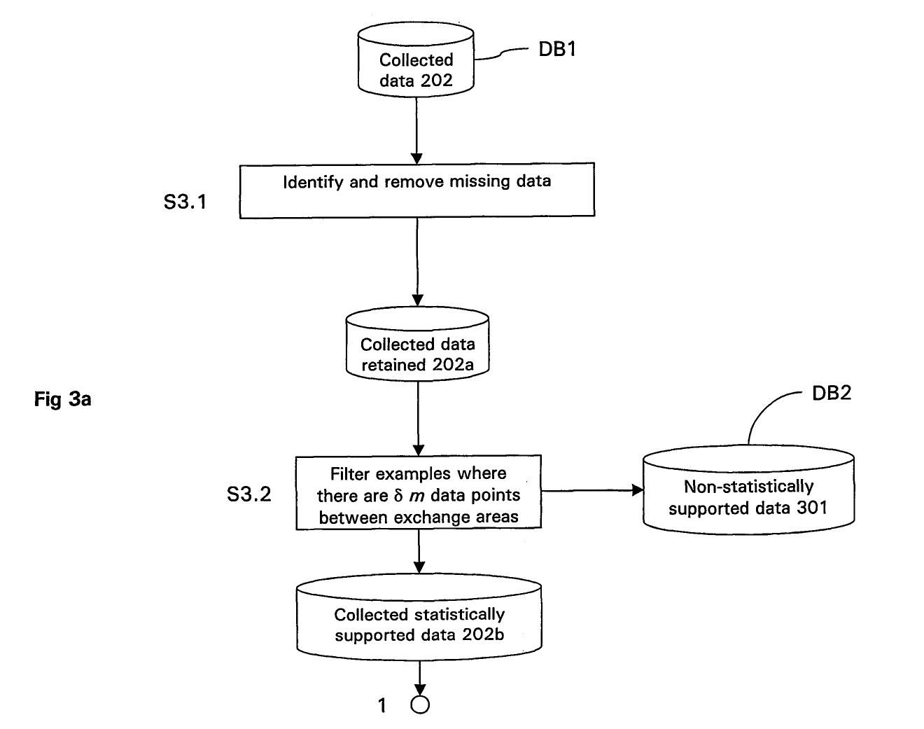

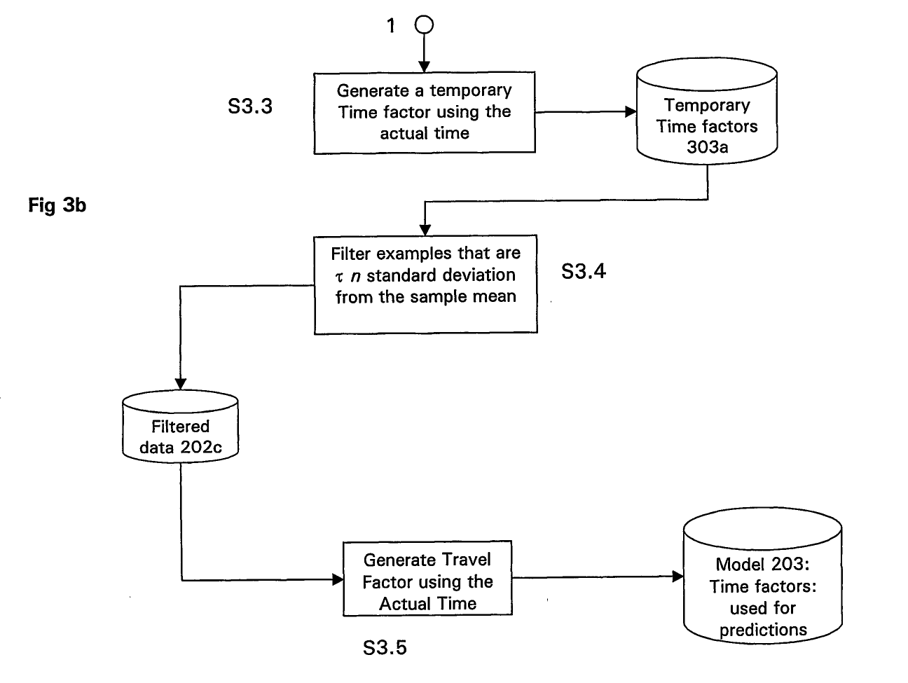

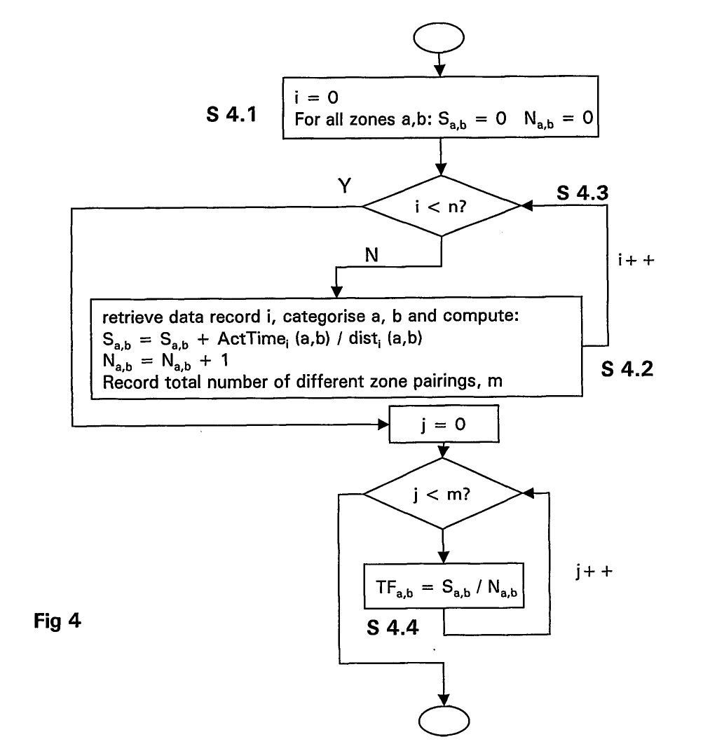

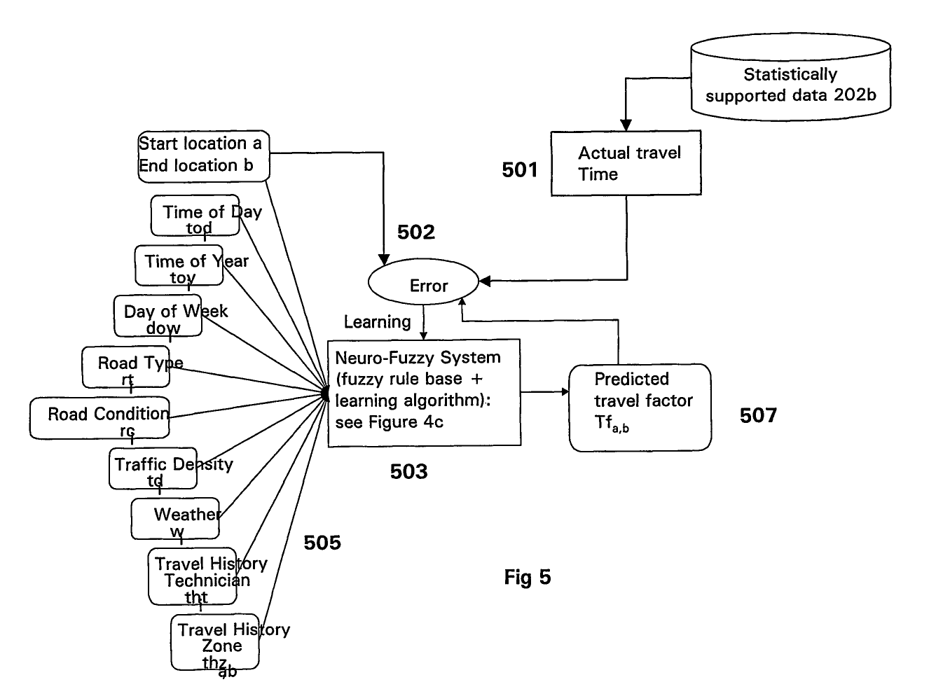

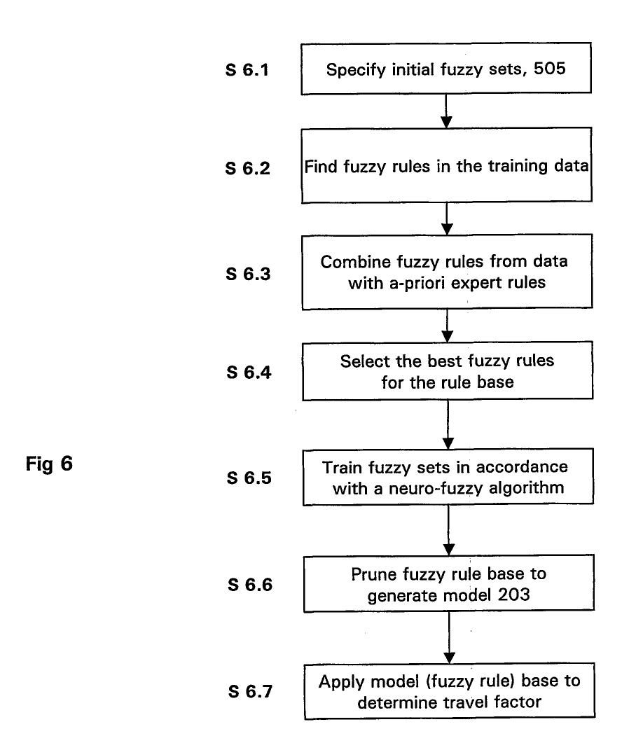

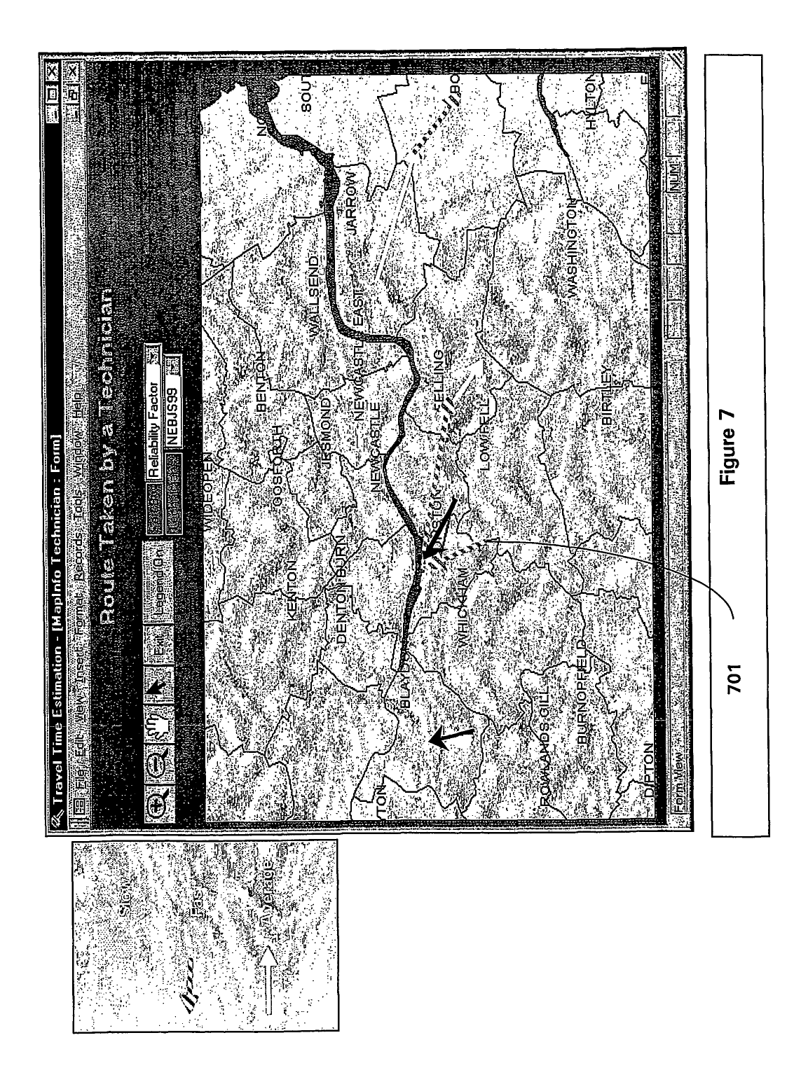

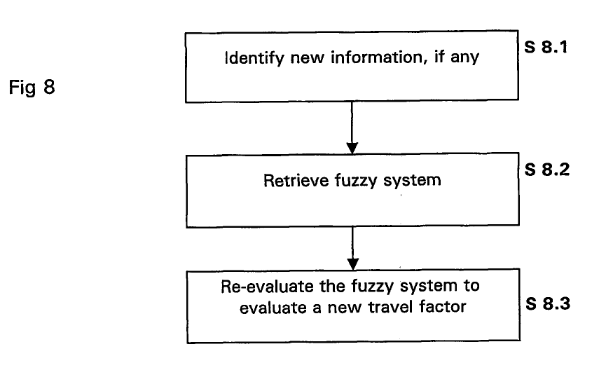

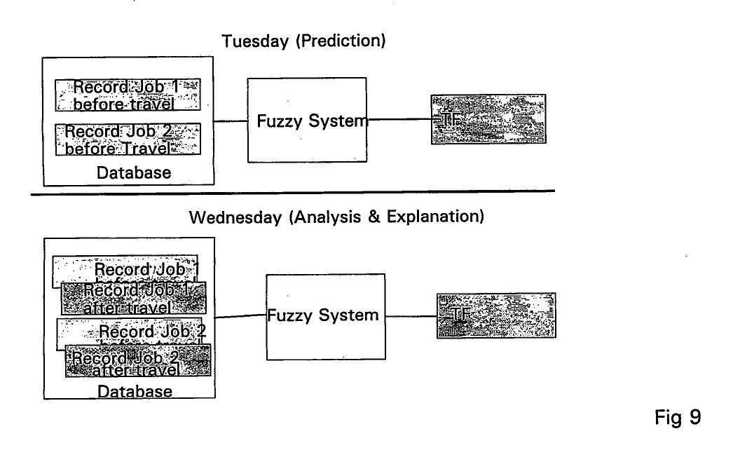

### FIELD OF THE INVENTION

The invention relates to a pharmacological substance based on biologically active substances obtained from the plant Tribulus terrestris L. to be used as an agent for reduction of blood sugar, improvement of blood circulation and especially blood circulation in veins and capillaries of the limbs of diabetic patients, reduction of bad cholesterol level, increase of good cholesterol concentration, maintenance of cardiovascular and liver activity with an additional prophylactic or healing effect on the immune system and immune resistance.

### PRIOR ART

Multiple agents and preparations for regulation of blood circulation, cardiovascular and liver activity as well as for cholesterol regulation of diabetics are known.

The disadvantages of the agents known lie in the necessity of complex use of different medical preparations in case the objective is to achieve simultaneous effects on different systems and organs of human body.

The use of trivalent chromium of different forms combined with other active substances representing extracts of the plant Ma Huang and coffein as an agent for overweight reduction is also known.

A Chinese medicine composition is known for curing ischemic encephalopathy, hyperlipemia, high blood viscosity, hypertension and diabetes with good therapeutic effect containing Chinese medicinal materials of American ginseng, earthworm, prepared flowery knotweed root, pueraria root, of tribulus terrestris, feted cassia seed, spanishneedles, epimedium, ligustrum fruit, aconus root, curcuma tuber and borneol and its preparation method includes the following steps: firstly freezing, drying and grinding earthworm, then grinding bomeol, and respectively sieving them; mixing the above-mentioned medicinal materials, decocting them by adding water twice, concentrating the decoction to obtain paste-like extract, cooling adding fine powders of the above mentioned other materials, uniformly stirring them so as to obtain the compound representing the invention.(CN1275388)

A disadvantage of the agent known is that it is difficult to prepare it and that it contains a complicated combination of biologically active substances of vegetal and animal origin the compositions of which are difficult to be controlled when rapid pharmacological effect is to be achieved.

### SUMMARY OF THE INVENTION

The purpose of the invention is to obtain a pharmacological substance representing a combination of bio-active substances of mineral and vegetal origin having a high biological capacity for stimulation and acceleration of blood circulation, limbs nutrition with blood and liver and cardiac activity in a natural way and for increasing diabetic's organism resistant forces.

The invention consists in making a pharmacological substance based on biologically active substances obtained from the plant Tribulus Terrestris L. and representing a combination of bio-active trivalent chromium of high glucoside-tolerance factor and a complex of natural products of biological-action steroid saponins obtained from Tribulus Terrestris l. and consisting of furostanols, spirostanols, sapogenins, sterols, flavonoids and other biologically active substances typical of this plant.

The complex of steroid saponins represents 40-80% by weight of the substance and contains furostanols, spirostanols sapogenins, sterols, flavonoids and other biologically active substances typical of this plant in the following rations in % by weight furostanols from 1 to 35%, spirostanols from 1 to 40%, sapogenins, from 0.01 to 30% sterols from 0.01% to 15%, flavonoids and other bio-active substances from 0.0001 to 15%.

The pharmacological substance contains a vegetal bio-active component representing a complex of steroid saponins obtained from the plant Tribulus Terrestris L., a mineral bio-active component and a pharmaceutical or nutritive base in the following ratios in % by weight: vegetal bio-active component representing a complex of steroid saponins obtained from the plant Tribulus Terrestris L. from 1 to 40%, mineral bio-active component representing a bio-active trivalent chromium from 0.000001 to 10% and a pharmaceutical or a nutritive base from 1 to 55%.

The pharmaceutical or nutritive base contains the following components in ratios in % by weight: micro-cristalline cellulose from 0.001 to 60%, dicalcium phosphate from 0.001 to 20%, stearic acid from 0 to 10%, magnesium stearate from 0.0001 to 10%, hydroxypropylic cellulose from 0 to 20% and highly dispersed silicon dioxide from 0.001 to 10%.

The pharmacological substance based on biologically active substances from the plant Tribulus Terrestris L. is obtained by the method where the drug or the extract from the plant Tribulus terrestris L. is subject to treatment at negative temperatures until obtaining a complex of steroid saponins containing furostanols, spirostanols, sapogenins, sterols, flavonoids and other biologically active substances typical of this plant in the following ratios in % by weight: furostanols from 1 to 35%, spirostanols from 1 to 40%, sapogenins, from 0.01 to 30% sterols from 0.01% to 15%, flavonoids and other bio-active substances from 0.0001 to 15%.

The pharmacological substance according to the invention is used for treatment or prophylaxis in case of high blood sugar values of diabetics and liver and cardiovascular diseases.

The Pharmacological Substance according to the invention is used for treatment or prophylaxis in case of inflammation of varicose veins, kidneys, bladder, prostate gland and as an agent of marked anti-inflammatory effect.

The Pharmacological Substance according to the invention is used for increasing good cholesterol concentration and reducing bad cholesterol concentration in blood.

The Pharmacological Substance according to the invention is used for treatment or prophylaxis as an active element of a solid medicine or as an addition to food.

The Pharmacological Substance according to the invention is simultaneously used for strengthening the immune system, increasing the immune resistance, regulation of the cardiovascular system and liver functioning.

The advantages of the invention are:

The Pharmacological Substance according to the invention is a bio-active complex of bio-active trivalent chromium of high glucoside-tolerance factor and high biological capacity obtained as a result of using special method of its production and of an enriched combination of furostanols, spirostanols, sapogenins, sterols and flavonoids obtained by a specific method of processing of a drug or extract from the leaves, stems and fruit of the plant Tribulus terrestris L. at negative temperatures.

The Pharmacological Substance according to the invention has an immediate effect on blood cells metabolism and bad and good cholesterol content in blood plasma while, in the absence of additional influences, having also effect on the correct functioning of blood circulation system, the cardiovascular activity and liver functioning of diabetics.

The Pharmacological Substance according to the invention contains a biologically active combination having a complex effect on blood cells metabolism, blood plasma content and cells biochemical composition as a whole, thus influencing at the same time blood circulation, the immune system and the correct metabolism of the whole organism.

The oral forms of the medicines and the additions to food made on the basis of the Pharmacological Substance according to the invention have a rapid action and are harmless in case of prolonged use in clinical conditions or at home. They are well tolerated by the organism, and they have no negative side effects.

### EXAMPLES

The following examples are illustrative of the invention but not limiting it:

### Example 1

Solid oral medicine including the Pharmacological Substance according to the invention and representing a tablet and containing the following components: 0.250 g vegetal active powder component made from leaves, stems and fruit of the plant Tribulus Terrestris L. and containing a complex of 50-80% by weight of steroid saponins, the content of the bio-active ingredients in % by weight being as follows:
furostanols from 20 to 30%
spirostanols from 30 to 35%
sapogenins from 17 to 25%
sterols from 0.01 to 10%
flavonoids and other active substances from 0.1 to 10%
   Mineral active component:
   bio-active trivalent chromium—0.0002 g
      Pharmaceutical base containing
monocrystalline cellulose—0.300 g
dicalcium phosphate—0.100 g
stearic acid—0.027 g
magnesium stearate—0.025 g
hydroxypropylic cellulose—0.020 g
highly dispersed silicon dioxide—0.001 g

### Example 2

Addition to food including the Pharmacological Substance according to the invention and representing a tablet and containing the following components: 0.250 g vegetal active component representing a total extract of leaves, stems and fruit of the plant Tribulus Terrestris L., containing a complex of 50-60% by weight of steroid saponins, the content of the bio-active ingredients in % by weight being as follows:
furostanols from 20 to 25%
spirostanols from 32 to 35%
sapogenins from 15 to 20%
sterols from 0.1 to 7%
flavonoids and other active substances from 0.1 to 12%
   Mineral active component:
   bio-active trivalent chromium—0.0002 g
      Nutritive base containing
monocrystalline cellulose—0.300 g
dicalcium phosphate—0.100 g
stearic acid—0.027 g
magnesium stearate—0.025 g
hydroxypropylic cellulose—0.020 g
highly dispersed silicon dioxide—0.001 g

### Example 3

Solid oral medicine including the pharmacological substance according to the invention and representing a gelatin capsule containing the following components: 0.250 g vegetal active powder from leaves, stems and fruit of the plant Tribulus Terrestris L., containing a complex of 50-80% by weight of steroid saponins, the content of bio-active ingredients in % by weight being as follows:
furostanols from 20 to 30%
spirostanols from 30 to 35%
sapogenins from 17 to 25%
sterols from 0.1 to 10%
flavonoids and other active substances from 0.1 to 10%
   Mineral active component:
   bio-active trivalent chromium—0.0002 g
      Pharmaceutical base containing
monocrystalline cellulose—0.350 g
dicalcilum phosphate—0.100 g
magnesium stearate—0.025 g
highly dispersed silicon dioxide—0.001 g

### Example 4

Addition to food including the pharmacological substance according to the invention and representing a solid gelatin capsule containing the following components: 0.250 g vegetal active component representing a total extract of leaves, stems and fruit of the plant Tribulus Terrestris L., containing a complex of 50-60% by weight of steroid saponins, the content of the bio-active ingredients in % by weight being as follows:
furostanols from 20 to 25%
spirostanols from 32 to 35%
sapogenins from 15 to 20%
sterols from 0.1 to 7%
flavonoids and other active substances from 0.1 to 12%
   Mineral active component:
   bio-active trivalent chromium—0.0002 g
      Nutritive base containing
monocrystalline cellulose—0.350 g
dicalcium phosphate—0.100 g
magnesium stearate—0.025 g
highly dispersed silicon dioxide—0.001 g

In another version of the invention the pharmacological substance and especially the complex of steroid saponins of its composition are obtained by processing or extracting at negative temperatures the drug obtained from Tribulus Terrestris L. collected in special conditions until obtaining an active component containing a complex of steroid saponins in a precise ratio in % by weight, this bio-active component being included as the basic vegetal active component of the compositions of the solid oral medicines prepared or of the additions to food.

### USE OF THE INVENTION

The medicine described in Example 1 has been clinically tested in the city of Plovdiv, Bulgaria on patients having increased values of blood sugar and bad cholesterol. This medicine has been administered according to an individual prescription for each case depending on the quantity of blood sugar in a daily dose of 2-6 tablets taken before meal, then blood sugar has been tested in the morning before having meal observing at the same time the parameters of bad and good cholesterol in blood. This medicine can be used by type 1 diabetics taking only tablets and by type 2 diabetics treated with insulin and controlling with difficulty their blood sugar or having a desire to pass to type 1. The dose has been fully assimilated by the organism and the result has become obvious on the very first day. Great tolerance and reliability have been observed, and the following pharmacological effects have been established:
high blood sugar level reduction by 30%;
bad cholesterol level reduction to the limit of its normal content as in healthy persons blood;
improvement of blood circulation in limbs capillaries and veins by 35%.

The addition to food described in Example 2 has been clinically tested in Santa Monica, USA, on diabetics showing high values of blood sugar and bad cholesterol. The addition has been administered according to an individual prescription in each case depending on the content of blood sugar and bad cholesterol in blood in daily doses 2-6 tablets, the following results having been observed:
high blood sugar level reduction by 50%;
improvement of cardiac and liver activity in 32% of the cases;
bad cholesterol level reduction to the limit of its normal content as in case of healthy persons blood;
good cholesterol level increase to the limit of its normal value as in healthy persons
anti-inflammatory effect on inflamed varicose veins, kidneys, bladder, prostate gland in 38% of the cases.

The medicine described in Example 3 has been clinically tested in Sofia, Bulgaria, during treatment of diabetics having high blood sugar level values and suffering from an inflammation of the prostate gland. The medicine has been administered according to a individual prescription in each case depending on blood sugar level and the degree of inflammation. Side effects have not been observed, the degree of tolerance by the patients has been very high, and the following results have been observed:
high blood sugar level reduction by 29%;
dying away of prostate gland inflammation by 43%.

The addition to food described in Example 4 has been tested in Merylville, USA, on diabetics having high blood sugar and poor blood circulation in limbs. The following results have been observed at the end of the treatment:
high blood sugar level reduction by 44%;
improvement of blood circulation in limbs by 67%; improvement and strengthening of the immune system and the immune resistance by 36%.

1. Pharmacological substance based on biologically active substances obtained from the plant Tribulus Terrestris L. characterized by that it is a combination of bio-active trivalent chromium and a complex of steroid saponins obtained from the plant Tribulus Terrestris L. and consisting of furostanols, spirostanols, sapogenins, sterols, flavonoids and other biologically active substances typical of this plant.

2. Pharmacological substance according to claim 1, characterized in that its complex of steroid saponins represents 40-80% by weight of the substance and contains furostanols, spirostanols sapogenins, sterols, flavonoids and other biologically active substances typical of this plant in the following ratios in % by weight:
furostanols from 1 to 35%,
spirostanols from 1 to 40%,
sapogenins, from 0.01 to 30%,
sterols from 0.01% to 15%, and
flavonoids and other bio-active substances from 0.0001 to 15%.

3. Pharmacological substance according to claim 1, characterized in that it contains a vegetal bio-active component representing a complex of steroid saponins obtained from the plant Tribulus Terrestris L., a mineral bio-active component and a pharmaceutical or nutritive base in the following ratios in % weight:
vegetal bio-active component representing a complex of steroid saponins obtained from the plant Tribulus Terrestris L. from 1 to 40%,
mineral bio-active component representing a bio-active trivalent chromium from 0.000001 to 10%, and
pharmaceutical or a nutritive base from 1 to 55%.

4. Pharmacological substance according to claim 3, characterized in that its pharmaceutical or nutritive base contains the following components in ratios in % by weight:
micro-cristalline cellulose from 0.001 to 60%,
dicalcium phosphate from 0.001 to 20%,
stearic acid from 0 to 10%,
magnesium stearate from 0.0001 to 10%,
hydroxypropylic cellulose from 0 to 20%, and
highly dispersed silicon dioxide from 0.001 to 10%.

5. A method of obtaining a pharmacological substance based on biologically active substances obtained from the plant Tribulus Terrestris L., characterized by processing an extract from the plant Tribulus Terrestris L. at negative temperatures until obtaining a complex of steroid saponins containing furostanols, spirostanols, sapogenins, sterols, flavonoids and other biologically active substances typical of this plant in the following ratios in % by weight:
furostanols from 1 to 35%,
spirostanols from 1 to 40%,
sapogenins, from 0.01 to 30%
sterols from 0.01% to 15%, and
flavonoids and other bio-active substances from 0.0001 to 15%.

6. A method for treatment or prophylaxis in case of high blood sugar values of diabetics and liver and cardiovascular diseases comprising administering the pharmacological substance according to claim 1 to a patient.

7. A method for treatment in case of inflammation at least one of varicose veins, kidneys, bladder, and prostate gland and for providing a marked anti-inflammatory effect comprising administering the pharmacological substance according to claim 1 to a patient.

8. A method for increasing good cholesterol concentration and reducing bad cholesterol concentration in blood comprising administering the pharmacological substance according to claim 1 to a patient.

9. A method for strengthening the immune system, increasing the immune resistance, regulation of the cardiovascular system and liver functioning comprising administering the pharmacological substance according to claim 1 to a patient.
